(19) United States
(12) Patent Application Publication
(10) Pub. No.: US 2005/0016539 A1
(43) Pub. Date: Jan. 27, 2005

(54) Closure device of a second-stage regulator for scuba divers

(75) Inventor: Stefano Pedemonte, Ceranesi (IT)

Correspondence Address: Grant E. Pollack, Esq.;Steinberg & Raskin, P.C., 15th Floor, 1140 Avenue of the Americas, New York, NY, 10036-5803, US

(73) Assignee: Cressi-Sub S.p.A.

(21) Appl. No.: 10/899,204

(22) Filed: Jul. 26, 2004

(30) Foreign Application Priority Data
Jul. 25, 2003 (IT) FI2003U000071

(51) Int. Cl. A61M 16/00

(52) U.S. Cl. 128/204.26

(57) ABSTRACT

A closure device of a second-stage regulator for scuba divers, the regulator comprising a regulator body with an inlet conduit for connection to a first-stage regulator which supplies a breathable gaseous mixture at relatively constant pressure, an outlet conduit for connection to a mouthpiece of the users, and an opening closed by a deformable diaphragm. The device comprises a locking frame connected jointedly to the regulator body to lock the diaphragm in the opening, and a member for fastening the frame to the regulator body.

### DESCRIPTION

1. Field of the Invention

The present invention relates generally to diving equipment and more particularly refers to a device for the closure of a second-stage regulator for scuba divers.

2. Background Art

It is known that the supply of air, or of the air-oxygen mixture, which is fed to the mouthpiece of the scuba diver from a high-pressure tank, passes via a first-stage pressure-reducing regulator to a second-stage regulator which supplies the mixture to the mouthpiece of the scuba diver when pressure within the regulator is diminished by a diver's inhalation.

Second-stage regulators of the known type have an inlet chamber connected to the outlet of the first-stage regulator, and an outlet chamber connected to the mouthpiece of the user and separated from the outside environment by an elastically deformable diaphragm which blocks an opening formed in the regulator body. The diaphragm is connected via a lever to a poppet which closes off the passage between the two chambers. The pressure inside the inlet chamber is maintained constant at approximately ten bars as the pressure in the tank varies thanks to appropriate calibration of the first-stage regulator. When the user does not breathe, his or her lungs, the mouthpiece, the outlet chamber and the outside environment are at the same pressure. When the user inhales, a vacuum is created inside the outlet chamber and the diaphragm bends towards the interior of said chamber moving the poppet, which normally closes the passage between the inlet chamber and the outlet chamber, to an opening position.

The opening of the passage between the inlet chamber and outlet chamber creates an overpressure in the outlet chamber, so that the diaphragm returns into the rest position, moving the lever and returning the poppet into the starting position wherein the passage between the inlet chamber and the outlet chamber is closed once again.

In second-stage regulators of the known type the diaphragm is locked at the edge of the opening formed in the regulator body by means of a screw cover in the case wherein the diaphragm and corresponding opening are circular in shape or, in the case of diaphragms and openings with a different shape, by attaching the cover to the regulator body by screws. This second solution is not considered satisfactory, above all for production reasons, in that it requires the provision of metal bushings inside the regulator body to be used as seats for the screws and, during assembly, the tightening of several screws. On the other hand the need to increase the size of the diaphragm as far as possible in order to reduce the effort required of the scuba diver during inhalation, in so far as the overall dimensions of the regulator allows, directs to the use of diaphragms with a shape other than the circular type, in particular diaphragms with an oval shape, with the consequent need of having to provide an adequate system for locking the diaphragm and closing the regulator.

### OBJECTS AND SUMMARY OF THE INVENTION

The object of the present invention is to provide a closure device of a second-stage regulator for scuba diver which enables the disadvantages indicated above to be avoided.

A particular object of the present invention is to provide a closure device of a second-stage regulator for scuba diver of the above mentioned type which allows locking of the diaphragm without the use of screws connections and also ensuring stability of the connection against any risk of accidental opening.

These objects are achieved with the closure device of a second-stage regulator for scuba diver the main features of which are disclosed in claim 1.

### BRIEF DESCRIPTION OF THE DRAWINGS

Further important features of the invention are now to be illustrated in greater detail by the following description of one of its embodiments, given by way of a non-limiting example with reference to the accompanying drawings, wherein:

FIG. 1 is a front perspective view of a second-stage regulator for scuba diver with the closure device according to the invention mounted thereon;

FIG. 2 is a side view of the second-stage regulator of FIG. 1;

FIG. 3 is a partially exploded side view of the regulator of FIG. 1 with the closure device in its open position;

FIG. 4 is a perspective view of the second-stage regulator of FIG. 1 wherein the means for locking the closure device are shown;

FIG. 5 is a side view of the means for locking the closure device;

FIG. 6 is an isometric view of the locking means of FIG. 5.

### DETAILED DESCRIPTION OF THE INVENTION

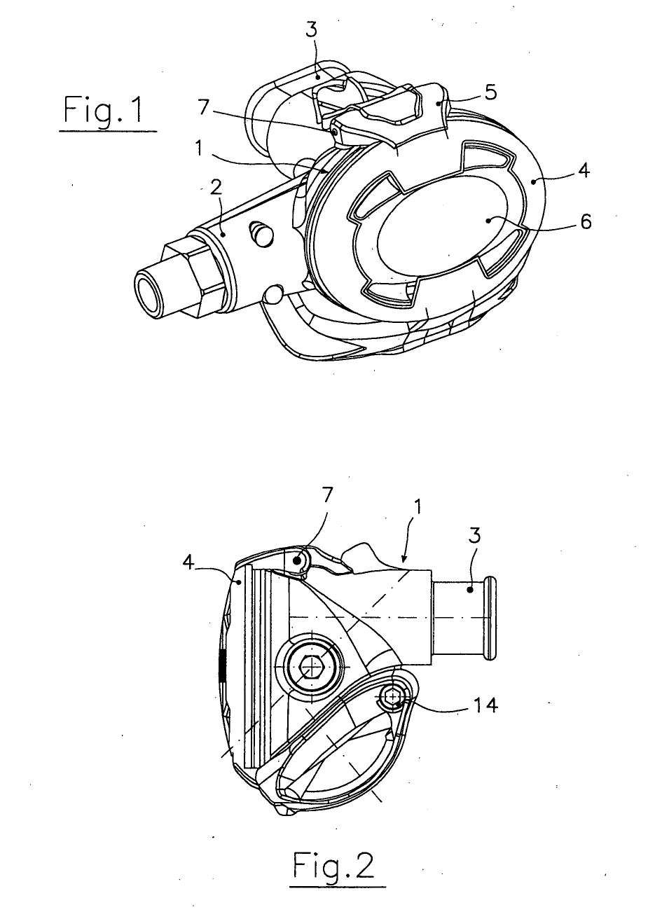

Referring to FIG. 1, 1 denotes the body of a second-stage regulator for scuba diver comprising an inlet conduit 2 and outlet conduit 3. The inlet conduit 2 is used for connection to a first-stage regulator which supplies a breathable gaseous mixture at constant pressure, while the outlet conduit 3 is designed to be held in the mouth by the scuba diver via a mouthpiece.

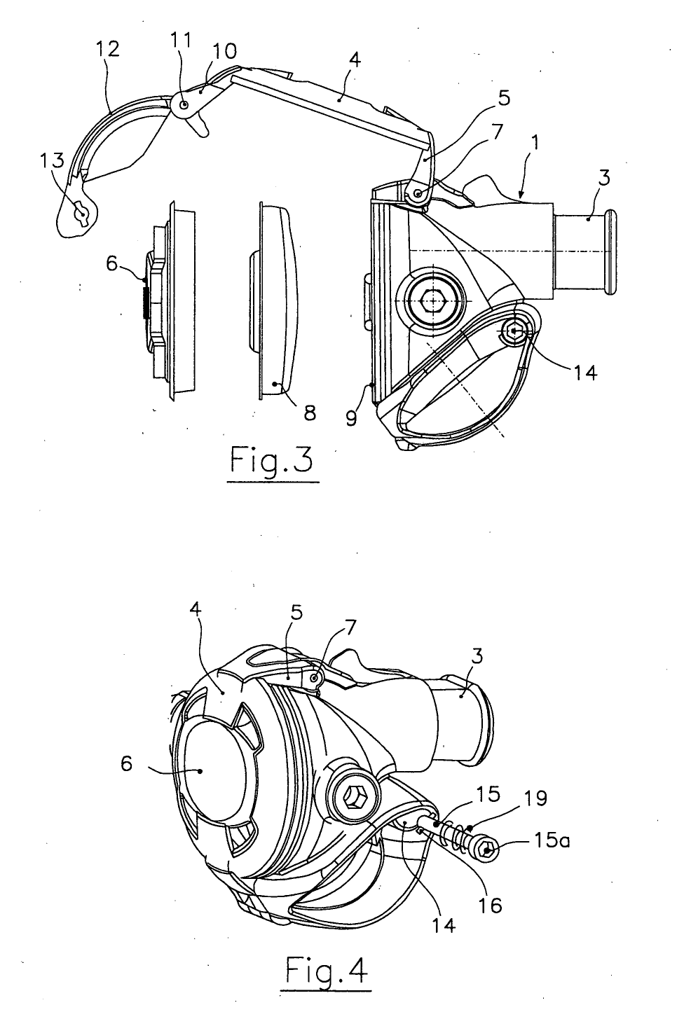

As shown also in FIGS. 2 and 3, the second-stage regulator comprises a front frame 4 with an arm 5 extending radially therefrom. Arm 5 is hinged to the regulator body 1 through a pin 7. A pushbutton 6 is provided centrally on the frame 4 to control the internal diaphragm 8, which can be seen in FIG. 3. The frame 4 acts as a cover and is used to lock the edges of the diaphragm 8 against the edges of an opening 9 formed in the regulator body 1.

The frame 4 has, at the end diametrically opposed to the arm 5, an additional arm 10 hinged through a pin 11 to a bracket 12. An elongated through hole 13 is formed in the free end of bracket 12, designed to align with a corresponding hole 14 of the regulator body when the frame is in the closure position shown in FIG. 4. In this position a pin 15 with a substantially T-shaped enlarged end 16 allows locking of the device above the regulator body 1.

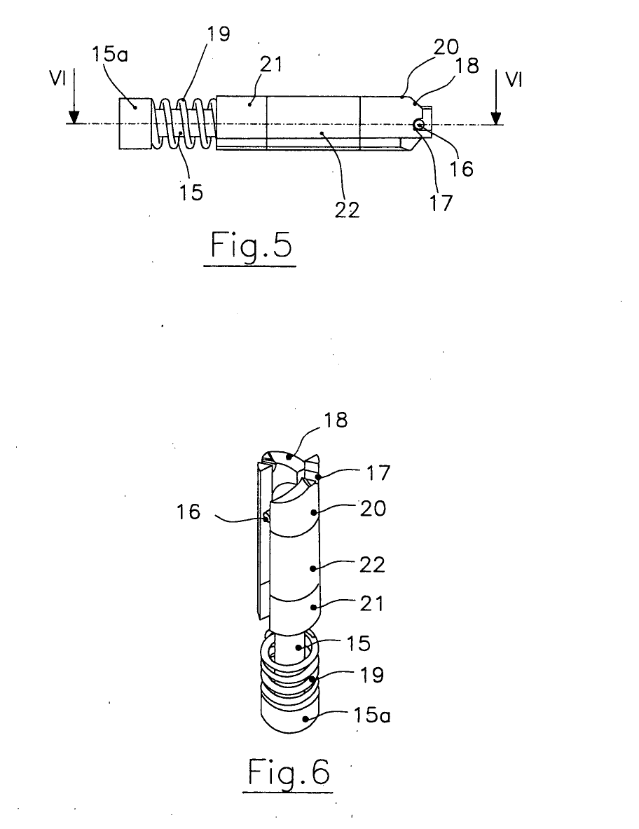

As shown in FIGS. 4, 5 and 6, the enlarged head 16 of the pin 15 can consist of a cross member which engages a seat 17 placed at the end of a cam profile 18. A spring 19, coaxial to the pin 15, maintains the cross member 16 in an engaged position elastically in the seat 17. In FIGS. 5 and 6 it should be noted that the elements denoted by 20 and 21 are integral with the regulator body 1, while the element 22 belongs to the end of the bracket 12. Accidental or unwanted opening of the regulator with release of the bracket 12 and of the frame 4 and consequent detachment of the diaphragm 8 is prevented by the fact that the pin 15 has a head 15a with a prismatic cavity of such a shape as to require the use of a special key for its operation. By compressing the head 15a it is possible to disengage the cross member of the enlarged head 16 of the pin 15 from the seat 17 and, following rotation through 90°, it is possible to slide it along the cam 18 to pass it through the elongated hole 13 of the bracket 12 thus allowing its withdrawal.

It should however be noted that the locking device formed by the pin 15 is a safety device in that the frame 4 is snap-fastened on the regulator body 1 via the bracket 12 and its relative end with elongated hole 13 and therefore the frame 4, once brought into the position of FIG. 4, is already in itself stably fastened on the regulator body 1.

The solution proposed with the present invention allows the transverse dimension of the regulator to be maintained within the overall dimensions of the apparatus which surrounds it, increasing however the surface of the diaphragm which can be made in an elliptical shape, with a clear advantage for the user, who is required to make a smaller effort in that the vacuum which has to be developed for inhalation is smaller to the extent the surface of the diaphragm is larger.

On the other hand the elliptical shape allows a lowering, still parallel, of the diaphragm when the vacuum caused by the act of inhalation is generated inside the regulator.

1 A closure device of a second-stage regulator for scuba divers, the regulator comprising a regulator body with an inlet conduit for connection to a first-stage regulator which supplies a breathable gaseous mixture at constant pressure, an outlet conduit for connection to a mouthpiece of the user, and an opening closed by a deformable diaphragm, the device comprising a locking frame connected jointedly to the regulator body to lock the diaphragm in the opening, a member being provided fastening the frame to the regulator body.

2 The closure device set forth in claim 1, wherein the frame is connected jointedly to a bracket which can be snap-attached to the regulator body with its free end.

3 The closure device set forth in claim 1, wherein the member for attaching the frame to the regulator body comprises a pin with an enlarged substantially T-shaped end, passing through the free end of the bracket for snap-engagement with a seat of the regulator body as a result of its rotation around its longitudinal axis, an elastic member being provided coaxially to the pin, for opposing disengagement of the enlarged end from the seat.

4 The closure device set forth in claim 3, wherein the seat of the regulator body is formed between two symmetrical cam profiles acting as a guide for the enlarged end of the pin when, as a result of axial angular displacement of the latter, the enlarged end moves from a position of free axial sliding to a position of engagement within the seat.

5 The closure device set forth in claim 4, wherein the pin has a head with a generally prismatic seat for device operation.
